(19) United States
(12) Patent Application Publication
(10) Pub. No.: US 2006/0057190 A1
(43) Pub. Date: Mar. 16, 2006

(54) Silicone topcoat with improved dirt repellency and improved bondability

(75) Inventor: Julia Henn, Mehring (DE)

Correspondence Address: BROOKS KUSHMAN P.C., 1000 TOWN CENTER, TWENTY-SECOND FLOOR, SOUTHFIELD, MI, 48075, US

(73) Assignee: Wacker-Chemie GmbH, Munich (DE)

(21) Appl. No.: 11/221,197

(22) Filed: Sep. 7, 2005

(30) Foreign Application Priority Data
Sep. 14, 2004 (DE) 10 2004 044 392.0

(51) Int. Cl. A61K 9/70

(52) U.S. Cl. 424/443

(57) ABSTRACT

A composition preparable using polymer components (1) (A1) polyorganosiloxanes comprising T and optionally M units and/or
(A2) polyorganosiloxanes comprising Q and optionally M units, with the proviso that per molecule there are 0.01% to 3.0% by weight of Si-bonded radicals OR¹, and also, optionally, one or more vinyl chloride-hydroxypropyl acrylate copolymers, vinyl acetate-ethylene copolymers, polyvinyl chloride, polyamides, polyesters, acrylate-polyester copolymers, polyamide-polyester copolymers, vinyl acetate-polyester copolymers, or monomeric (meth)acrylates, the (meth)acrylates copolymerized with silanes containing Si-bonded (meth)acrylate groups, (2) silane(s)
   R³ₓSi(OR²)₄₋ₓ, and (3) crosslinked silicone particles composed of a single molecule which have an average diameter of 5 to 200 nm, are useful for forming coatings which bond well to substrates, exhibit low friction, and are dirt repellant.

### BACKGROUND OF THE INVENTION

1. Field of the Invention

The invention relates to a polyorganosiloxane composition and also to shaped articles, preferably sheetlike structures or elastomers prepared therefrom.

2. Background Art

EP 718 355 A discloses compositions comprising polyorganosiloxane components which even as of its filing date demonstrated improved dirt repellency in relation to the then-known state of the art. However, the bondability of coatings comprising this composition to a silicone substrate is poor.

### SUMMARY OF THE INVENTION

It is an object of the invention to improve on the known state of the art, and in particular to improve further the dirt repellency and the bondability of organopolysiloxanes, preferably used as topcoats. These and other objects have been achieved through use of a composition containing an organopolysiloxane resin bearing a limited quantity of alkoxy groups, an alkoxysilane, and solid organopolysiloxane particles, each particle being a single molecule.

### DETAILED DESCRIPTION OF THE PREFERRED EMBODIMENT(S)

The invention provides a composition preparable using, as polymer components (1)
(A1) polyorganosiloxanes comprising units (T units) of the formula (R1Si—O_{3/2}) and optionally units (M units) of the formula (R₃Si—O_{1/2}) and/or
(A2) polyorganosiloxanes comprising units (Q units) of the formula (Si—O_{4/2}) and optionally units (M units) of the formula (R₃Si—O_{1/2}) in which R, identical or different at each occurrence, denotes unhalogenated or halogenated (“optionally halogenated”) hydrocarbon radicals having 1-18 carbon atoms per radical or denotes OR¹ where R¹ can be identical or different at each occurrence and denotes hydrogen or a monovalent, unsubstituted or substituted (“optionally substituted”) hydrocarbon radical having 1-8 carbon atom(s), with the proviso that per molecule there are 0.01% to 3.0% by weight of Si-bonded radicals OR¹, and also, optionally, one or more polymer components selected from the group consisting of:
(B) vinyl chloride-hydroxypropyl acrylate copolymers,
(C) vinyl acetate-ethylene copolymers,
(D) polyvinyl chloride,
(E) polyamides,
(F) polyesters,
(G) acrylate-polyester copolymers,
(H) polyamide-polyester copolymers,
(I) vinyl acetate-polyester copolymers, and
(J) monomeric (meth)acrylates, with the proviso that the monomeric (meth)acrylates are copolymerized with silanes containing Si-bonded (meth)acrylate groups,
(2) at least one silane of the general formula
   R³ₓSi(OR²)₄₋ₓ
   where R² is a monovalent, unsubstituted or substituted hydrocarbon radical,
   R³ is a monovalent organic radical,
   x is 0 or 1,
(3) silicone particles,
(4) optionally, solvent,
(5) optionally, catalyst, and
(6) optionally, water.

Examples of radicals R are preferably alkyl radicals such as the methyl, ethyl, n-propyl, isopropyl, n-butyl, isobutyl, tert-butyl, n-pentyl, isopentyl, neopentyl, and tert-pentyl radicals; hexyl radicals such as the n-hexyl radical; heptyl radicals such as the n-heptyl radical; octyl radicals such as the n-octyl radical and isooctyl radicals such as the 2,2,4-trimethylpentyl radical; nonyl radicals such as the n-nonyl radical; decyl radicals such as the n-decyl radical; dodecyl radicals such as the n-dodecyl radical; octadecyl radicals such as the n-octadecyl radical; alkenyl radicals such as the vinyl and the allyl radicals; and cycloalkyl radicals such as the cyclopentyl, cyclohexyl, cycloheptyl, and methylcyclohexyl radicals.

Examples of substituted radicals R are cyanoalkyl radicals such as the P-cyanoethyl radical, and halogenated hydrocarbon radicals, examples being haloalkyl radicals, such as the 3,3,3-trifluoro-n-propyl radical, the 2,2,2,2′,2′,2′-hexafluoroisopropyl radical, and the heptafluoroisopropyl radical.

Simply for reasons of easier availability, methyl and ethyl radicals are preferred as radicals R.

Radical R¹ preferably comprises a hydrogen atom or an unsubstituted or substituted (“optionally substituted”) hydrocarbon radical having 1 to 8 carbon atom(s), particular preference being given to hydrogen and alkyl radicals having 1 to 3 carbon atom(s), especially the methyl, ethyl, and isopropyl radicals. Examples of radicals R¹ include the examples as stated for the radical R but have 1 to 8 carbon atom(s).

Examples of radicals R² are preferably unsubstituted or substituted hydrocarbon radicals having 1-18 carbon atom(s), particular preference being given to alkyl radicals such as the methyl, ethyl, n-propyl, isopropyl, n-butyl, isobutyl, tert-butyl, n-pentyl, isopentyl, neopentyl, and tert-pentyl radicals; hexyl radicals such as the n-hexyl and isohexyl radicals; heptyl radicals such as the n-heptyl and isoheptyl radicals; octyl radicals such as the n-octyl radical and isooctyl radicals such as the 2,2,4-trimethylpentyl radical. Preference is given to the methyl and ethyl radicals. Examples of hydrocarbon radicals R² which may be substituted by an ether oxygen atom are the methoxyethyl, the ethoxyethyl, the methoxy-n-propyl and the methoxyisopropyl radicals.

Examples of radicals R³ are preferably unsubstituted or substituted hydrocarbon radicals having 1-18 carbon atom(s), particular preference being given to alkyl radicals such as the methyl, ethyl, n-propyl, isopropyl, n-butyl, isobutyl, tert-butyl, n-pentyl, isopentyl, neopentyl, and tert-pentyl radicals; hexyl radicals such as the n-hexyl and isohexyl radicals; heptyl radicals such as the n-heptyl and isoheptyl radicals; octyl radicals such as the n-octyl radical and isooctyl radicals such as the 2,2,4-trimethylpentyl radical; nonyl radicals such as the n-nonyl and isononyl radicals; decyl radicals such as the n-decyl and isodecyl radicals; dodecyl radicals such as the n-dodecyl and isodecyl radicals; octadecyl radicals such as the n-octadecyl and isooctadecyl radicals; alkenyl radicals such as the vinyl and the allyl radical; cycloalkyl radicals such as the cyclopentyl, cyclohexyl, cycloheptyl and methylcyclohexyl radicals; aryl radicals such as the phenyl, naphthyl, anthryl and phenanthryl radicals; alkaryl radicals such as the o-, m-, and p-tolyl radicals, xylyl radicals, ethylphenyl radicals, o-, m-, and p-vinylphenyl radicals, and the nonylphenyl radical; and aralkyl radicals such as the benzyl radical, the α- and the β-phenylethyl radicals; isocyanatoalkyl radicals such as the isocyanatopropyl, isocyanatoethyl, isocyanatohexyl, and isocyanatooctyl radicals, the isocyanatopropyl radical being preferred, and (meth)acryloyloxy radicals such as the methacryl-oyloxypropyl, acryloyloxypropyl, methacryloyloxyhexyl, and acryloyloxyhexyl radicals, the methacryloyloxypropyl radical being preferred.

Examples of halogenated hydrocarbon radicals R are haloalkyl radicals such as the 3-chloro-n-propyl radical, the 3,3,3-trifluoro-n-propyl radical, the 2,2,2,2′,2′,2′-hexafluoroisopropyl radical, the hepta-fluoroisopropyl radical, and haloaryl radicals such as the o-, m-, and p-chlorophenyl radicals.

In the polyorganosiloxanes (A1) the ratio of M units to T units is 0 to 1.8:1, preferably 0.1 to 1.2:1 and more preferably 0.3 to 0.8:1, and in the polyorganosiloxanes (A2) the ratio of M units to Q units is 0.00 to 2.7:1, preferably 0.01 to 2.1:1, more preferably 0.1 to 1.8:1. The polyorganosiloxanes (A1) and (A2) of the invention form a polymer composed of 2-500, preferably 4-300, monomer units.

The polymer components (A1) and (A2) can be used alone or in each case as mixtures or reaction products of the organosiloxane units, preferably in a ratio of 1:20 to 20:1, more preferably 1:10 to 10:1. Preference is given to polymer components, such as resin solution K or resin solution K 0118 from Wacker-Chemie GmbH. These resins preferably are in solution in solvents, such as toluene, xylene, acetone, ethyl acetate, and ethanol. The solvents are preferably used in amounts of 10% to 98% by weight, preferably 30% to 98% by weight, based in each case on the total weight of the polymer components.

Besides the polymer components (A1) and (A2) it is also possible as polymer components (B) to use polymers such as vinyl chloride-hydroxypropyl acrylate copolymers. Products of this kind are offered commercially by Vinnolit GmbH under the name Vinnolit E 15/40 A. Copolymers (C) of vinyl acetate and ethylene can likewise be employed as polymer components. Using methods which are known in the literature, both monomers can be used to prepare copolymers in any desired ratio. As further polymer component it is possible to use (D) polyvinyl chloride, (E) polyamides, (F) polyesters, (G) acrylate-polyester copolymers, (H) polyamide-polyester copolymers, or (I) vinyl acetate-polyester copolymers, or (J) monomeric (meth)acrylates, such as methyl methacrylate or butyl methacrylate, which are polymerized in the reaction mixture. Preference is given to polymer components (A1), (A2), (B), and (C), particular preference to the polymer components (A1) and (A2) alone.

The polymer components are present in amounts of 2%-70% by weight in the compositions of the invention. A preferred amount is 5%-50% by weight, a more preferred amount being 10%-40% by weight.

Preferred examples of silanes (2) are methacryl-oyloxypropyltrimethoxysilane (trade name Silan GF 31—Wacker-Chemie GmbH), methyltriethoxysilane (trade name Silan M1-Triethoxy—Wacker-Chemie GmbH), vinyltriethoxysilane (trade name Silan GF 56—Wacker-Chemie GmbH), tetraethoxysilane (trade name TES 28—Wacker-Chemie GmbH), mixtures of low molecular mass hydrolysis products of tetraethoxysilane (trade name TES 40—Wacker-Chemie GmbH), methyltrimethoxysilane (trade name M1-Trimethoxy—Wacker-Chemie GmbH), and isocyanatopropyltrimethoxysilane (trade name Silan Y 9030 UCC).

The silanes are present in amounts of 0.1%-20% by weight, preferably of 0.5%-10% by weight, and the polymer components are preferably used with the silanes (2) or mixtures thereof in a ratio of 100:1 to 100:30, more preferably 100:2 to 100:20.

The compositions are preferably prepared in organic solvents such as tetrahydrofuran, toluene, acetone, naphtha, petroleum spirit, methyl ethyl ketone, xylene, butyl alcohol, ethyl acetate, isopropyl acetate or isopropanol. Organic solvents are present in amounts of 10% to 90% by weight, preference being given to 30%-85% by weight.

The compositions are mixed, when desired, with condensation catalysts, preferably organic tin compounds or organic zirconium compounds, preferred tin and zirconium compounds being zirconium butoxide, dibutyltin dilaurate, dibutyltin oxide, dioctyltin dilaurate, and dibutyltin diacetate. Among these condensation catalysts preference is given to dibutyltin dilaurate, dibutyltin acetate, and zirconium butoxide. The condensation catalysts are present in amounts between 0-10% by weight. Preference is given to amounts of 0-5% by weight, with particular preference given to amounts of 0-2% by weight.

A preferred source of free radicals, which are preferably used in connection with the polymer component (J) are peroxides, especially organic peroxides. Examples of such organic peroxides are peroxyketals, e.g., 1,1-bis(tert-butylperoxy)-3,3,5-trimethylcyclohexane and 2,2-bis(tert-butylperoxy)butane, and diacyl peroxides such as acetyl peroxide, isobutyl peroxide, benzoyl peroxide, and the like, dialkyl peroxides such as di-tert-butyl peroxide, tert-butyl cumyl peroxide, dicumyl peroxide, 2,5-dimethyl-2,5-di(tert-butylperoxy)hexane, tert-butyl perethylhexanoates, and the like, and peresters such as tert-butyl peroxyisopropyl carbonate. Preference is given to t-butyl perethylhexanoates (trade name Peroxan PO); Interox TBPIN.

Peroxides are preferably used in amounts of 0 to 5% by weight, in particular of 0 to 3% by weight, based in each case on the weight of the compound employed in the process of the invention.

Where appropriate it is also possible to add water in amounts of 0-20% by weight, preferably 0-10% by weight.

The compositions are composed either of silicone resins, which are mixed with functional silanes and hydrolyzed in organic solvents, or of organic (co)polymers, which are copolymerized with functional silanes.

The silicone particles are crosslinked organopolysiloxane particles which are composed of a single molecule and which have an average diameter of 5 to 200 nm, with at least 80% of the particles possessing a diameter which deviates by not more than 30% from the average diameter, these particles being soluble to an extent of at least 5% by weight in a solvent.

The organopolysiloxane particles typically have average molar masses of at least 10⁵, in particular 5×10⁵ to a maximum of 10¹⁰, in particular 10⁹. The average diameters of the organopolysiloxane particles are preferably at least 10 and preferably not more than 150 nm. Preferably at least 80% of the particles possess a diameter which deviates by not more than 20%, in particular not more than 10%, from the average diameter. The organopolysiloxane particles are preferably spherical microgels.

The organopolysiloxane particles are intramolecularly crosslinked but exhibit no intermolecular crosslinking between the organopolysiloxane particles. Consequently the organopolysiloxane particles are readily soluble in solvents. The solvent in which the organopolysiloxane particles dissolve to an extent of at least 5% by weight depends on the structure of the organopolysiloxane particles and in particular on the groups located on the surface of the organopolysiloxane particles. For all organopolysiloxane particles there is a suitable solvent. Examples of such solvents are water; alcohols such as methanol, ethanol, n-propanol, and isopropanol; ethers such as dioxane, tetrahydrofuran, diethyl ether and diethylene glycol dimethyl ether; chlorinated hydrocarbons such as dichloromethane, trichloromethane, tetrachloromethane, 1,2-dichloroethane, and trichloroethylene; hydrocarbons such as pentane, n-hexane, cyclohexane, hexane isomer mixtures, heptane, octane, universal spirits, petroleum ether, benzene, toluene, and xylenes; ketones such as acetone, methyl ethyl ketone, and methyl isobutyl ketone; dimethylformamide, carbon disulfide, and nitrobenzene, or mixtures of these solvents, and also monomers such as methyl methacrylate or styrene, and polymers, such as liquid organopolysiloxanes.

The solubility of the organopolysiloxane particles can be determined at 20° C. A particularly suitable solvent for organopolysiloxane particles having hydrocarbon radicals is toluene; for organopolysiloxane particles having amino radicals a particularly suitable solvent is tetrahydrofuran; and for organopolysiloxane particles having sulfonato radicals a particularly suitable solvent is water. By way of example, in toluene, organopolysiloxane particles having hydrocarbon radicals are of almost infinite solubility and in liquid polydimethylsiloxane with a viscosity of 35 mPa.s at 25° C. they are soluble to an extent of 15% by weight. Preferably the organopolysiloxane particles are at least 10% by weight soluble, in particular at least 15% by weight soluble, in a solvent selected from toluene, tetrahydrofuran, and water.

The organopolysiloxane particles are preferably composed of 0.5% to 80.0% by weight of units of the general formula
[R⁴₃SiO_{1/2}] (1),
0 to 99.0% by weight of units of the general formula
[R⁴₂SiO_{2/2}] (2),
0 to 99.5% by weight of units of the general formula
[R⁴SiO_{3/2}] (3),
0 to 80.0% by weight of units of the general formula
[SiO_{4/2}] (4), and
0 to 20.0% by weight of units of the general formula
[R⁴ₐSi(O_{(3-a)/2})—R⁵—X—(R ⁵Si(O_{(3-a)/2}))_{b}R⁴ₐ] (5),
where
R⁴ denotes a hydrogen atom or identical or different monovalent SiC-bonded, optionally substituted C₁ to C₁₈ hydrocarbon radicals,
R⁵ denotes identical or different divalent SiC-bonded, optionally substituted C₁ to C₁₈ hydrocarbon radicals, which may be interrupted by divalent radicals attached on both sides to carbon atoms and selected from the group consisting of —O—, —COO—, —OOC—, —CONR⁶—, —NR⁶CO—, and —CO—,
R⁶ is a hydrogen atom or a radical R⁴,
X is a radical from the group consisting of —N═N—, —O—O—, —S—S—, and —C(C₆H₅)₂—C(C₆H₅)₂—,
a denotes the values 0, 1 or 2, and,
b denotes the values 0 or 1,
   with the proviso that the sum of the units of the general formulae (3) and (4) is at least 0.5% by weight.

Examples of unsubstituted radicals R⁴ are alkyl radicals such as the methyl, ethyl, n-propyl, isopropyl, n-butyl, isobutyl, tert-butyl, n-pentyl, isopentyl, neopentyl, and tert-pentyl radicals, hexyl radicals such as the n-hexyl radical, heptyl radicals such as the n-heptyl radical, octyl radicals such as the n-octyl radical and isooctyl radicals such as the 2,2,4-trimethylpentyl radical, nonyl radicals such as the n-nonyl radical, decyl radicals such as the n-decyl radical, dodecyl radicals such as the n-dodecyl radical, octadecyl radicals such as the n-octadecyl radical; alkenyl radicals such as the vinyl, allyl, n-5-hexenyl, 4-vinylcyclohexyl, and 3-norbornenyl radicals; cycloalkyl radicals such as cyclopentyl, cyclohexyl, 4-ethylcyclohexyl, and cycloheptyl radicals, norbornyl radicals, and methylcyclohexyl radicals; aryl radicals such as the phenyl, biphenylyl, naphthyl, anthryl, and phenanthryl radical; alkaryl radicals such as o-, m-, p-tolyl radicals, xylyl radicals, and ethylphenyl radicals; aralkyl radicals such as the benzyl radical, the α- and β-phenylethyl radicals, and also the fluorenyl radical.

Examples of substituted hydrocarbon radicals as radical R are halogenated hydrocarbon radicals such as the chloromethyl, 3-chloropropyl, 3-bromopropyl, 3,3,3-trifluoropropyl and 5,5,5,4,4,3,3-heptafluoropentyl radicals and also the chlorophenyl, dichlorophenyl and trifluorotolyl radicals; mercaptoalkyl radicals such as the 2-mercaptoethyl and 3-mercaptopropyl radicals; cyanoalkyl radicals such as the 2-cyanoethyl and 3-cyanopropyl radicals; aminoalkyl radicals, such as the 3-aminopropyl, N-(2-aminoethyl)-3-aminopropyl, and N-(2-aminoethyl)-3-amino-(2-methyl)propyl radicals; amino-aryl radicals such as the aminophenyl radical; quaternary ammonium radicals; acryloyloxyalkyl radicals such as the 3-acryloyloxypropyl and 3-meth-acryloyloxypropyl radicals; hydroxyalkyl radicals such as the hydroxypropyl radical; phosphonic acid radicals; and phosphonate radicals and sulfonate radicals such as the 2-diethoxyphosphonatoethyl radical or the 3-sulfonatopropyl radical.

The radical R⁴ preferably comprises unsubstituted and substituted C₁ to C₆ alkyl radicals, hydrogen, and the phenyl radical, in particular the methyl, phenyl, vinyl, allyl, methacryloyloxypropyl, 3-chloropropyl, 3-mercaptopropyl, 3-aminopropyl and (2-aminoethyl)-3-aminopropyl radicals, hydrogen, and quaternary ammonium radicals.

Examples of divalent hydrocarbon radicals R⁵ are saturated alkylene radicals such as the methylene and ethylene radical, and also propylene, butylene, pentylene, hexylene, cyclohexylene, and octadecylene radicals, or unsaturated alkylene or arylene radicals, such as the hexenylene and phenylene radicals, and, in particular, radicals of the formulae (6)
—(CH₂)₃N(R⁷)—C(O)—(CH₂)₂—C(CN)(CH₃)— (6),
in which
R⁷ denotes a hydrogen atom, a methyl or cyclohexyl radical and (7)
   —(CH₂)₃—O—C(O)—(CH₂)₂—C(O)— (7).

Preferred radicals X are —N═N— and —O—O—.

Particularly preferred units of the general formula (5) come under the general formula (8)
[(CH₃)ₐSi(O_{(3-a)/2})—(CH₂)₃—N(R⁷)—C(O)—(CH₂)₂—C(CN)(CH₃)—N═]₂
in which a and R⁷ are as defined above.

The organopolysiloxane particles preferably contain 1% to 80.0% by weight of units of the general formula (1), 0 to 98.0% by weight of units of the general formula (2), 0 to 99.0% by weight of units of the general formula (3), 0 to 50.0% by weight of units of the general formula (4), and 0 to 10.0% by weight of units of the general formula (5), with the proviso that the sum of the units of the general formulae (3) and (4) is at least 1% by weight.

In particular the organopolysiloxane particles contain 5% to 70.0% by weight of units of the general formula (1), 0 to 94.0% by weight of units of the general formula (2), 1% to 95.0% by weight of units of the general formula (3), 0% by weight of units of the general formula (4), and 0 to 5.0% by weight of units of the general formula (5). The preparation of the organopolysiloxane particles takes place preferably in accordance with EP 744 432 (Wacker-Chemie GmbH) and its examples.

Particularly suitable for the structural characterization of the organopolysiloxane particles are static and dynamic light scattering. Static and dynamic light scattering are established methods in macromolecular chemistry and colloid chemistry for characterizing dispersed particles, and are known to the skilled worker. In static light scattering the scattering intensity at different angles is averaged over a sufficiently long time interval and information is obtained about the static properties of the macromolecules, such as the weight-average molar mass M_{w}, the z-average of the square of the radius of gyration <R_{g}2>_{z}, and the second virial coefficient A₂, which describes the intramolecular and intermolecular thermodynamic interactions of the dispersed particles with the solvent. In contrast to static light scattering, in the case of dynamic light scattering the fluctuation of the scattered-light intensity is observed as a function of time. This provides information on the dynamic behavior of the molecules under investigation. Measurements are made of the z-average of the diffusion coefficient D_{z} and hence, via the Stokes-Einstein law, of the hydrodynamic radius Rh and the coefficient k_{d}, which describes the concentration dependence of the diffusion coefficient. From -the angular dependence of the scattered light it is possible to determine the particle shape and any structuring present in solution can be clarified. Simultaneous static and dynamic light scattering measurement makes it possible to obtain the abovementioned information on the system under investigation with a single experiment and hence to obtain data concerning, for example, particle size, particle dispersity, and particle shape, and also on molecular weight and density. This is described in, for example, M. Schmidt, Simultaneous Static and Dynamic Light Scattering: Applications to Polymer Structure Analysis, in: Dynamic Light Scattering: The Method and some Applications; Brown, W. (ed.); Oxford University Press, Oxford, UK, 372-406 (1993).

The quotient of radius of gyration and hydrodynamic radius, referred to as the ρ ratio, provides structural information on the particle shape, such as hard spheres, hollow spheres, coils, rods or star polymer. For the “hard sphere” particle shape the theoretical ρ ratio is 0.775; the values measured for the preferred organopolysiloxane particles lie from 0.775 to a maximum of ρ=1.0. The preferred organopolysiloxane particles are therefore spherical.

The size range of the organopolysiloxane particles represents the boundary region between large molecules, oligomers, and dendrimers on the one hand and small solid bodies on the other, and thus corresponds to an interface between solid body and molecule. On the one hand, collective solid-body properties have not yet developed; on the other hand, molecular behavior can no longer be observed, or can be observed only occasionally. Examples of particulate structures of this order of size, with virtually fixed conformation, are microgels. According to Antonietti (Angew. Chemie 100 (1988) 1813-1817) microgels obtained from aqueous colloidal systems and having particle diameters in the mesoscopic size range of 5 to 200 nm and molar masses of 106 to 1011 (g/mol) are referred to as “type B” microgels. “Type B” microgels are of particular interest, for example, as fillers or compatibilizers for (optically transparent) polymers or as potential starting materials for tailor-made catalyst systems.

The silicone elastomer particles preferably have a size of 80-120 nm and are in methyl isobutyl ketone. Other solvents such as toluene, acetone and ethyl acetate and butyl acetate are possible, but MIBK has proven particularly advantageous.

The invention further provides a process for preparing a composition, the components of the composition being reacted or mixed, where reaction or mixing take place using polymer components (1)
(A1) polyorganosiloxanes comprising units (T units) of the formula (R₁Si—O_{3/2}) and optionally units (M units) of the formula (R₃Si—O_{1/2}) and/or
(A2) polyorganosiloxanes comprising units (Q units) of the formula (S₁—O_{4/2}) and optionally units (M units) of the formula (R₃Si—O_{1/2}) in which
R, identical or different at each occurrence, denotes optionally halogenated hydrocarbon radicals having 1-18 carbon atoms per radical or denotes OR¹ where
R¹ can be identical or different at each occurrence and denotes hydrogen or a monovalent, optionally substituted hydrocarbon radical having 1-8 carbon atom(s),
   with the proviso that per molecule there are 0.01% to 3.0% by weight of Si-bonded radicals OR¹,
and also, optionally, one or more polymer components selected from the group consisting of:
   (B) vinyl chloride-hydroxypropyl acrylate copolymers,
   (C) vinyl acetate-ethylene copolymers,
   (D) polyvinyl chloride,
   (E) polyamides,
   (F) polyesters,
   (G) acrylate-polyester copolymers,
   (H) polyamide-polyester copolymers,
   (I) vinyl acetate-polyester copolymers, and
   (J) monomeric (meth)acrylates, with the proviso that the latter are copolymerized with silanes containing Si-bonded (meth)acrylate groups,
   (2) silane of the general formula
      R³ₓSi(OR²)₄₋ₓ
      where R² is a monovalent, unsubstituted or substituted hydrocarbon radical, R³ is a monovalent organic radical,
x is 0 or 1,
(3) silicone particles,
(4) optionally, solvent,
(5) optionally, catalyst, and
(6) optionally, water.

Examples of R, R¹, R², and R³ are the examples given above for the radicals R, R¹, R², and R³.

The compositions of the invention can be prepared in stirring and mixing units such as are customary in the chemical industry. The units are desirably temperature-controllable in the range from −10° C. to +150° C. Owing to the use of organic solvents, measures to prevent explosion are vital.

The compositions are prepared simply by mixing the individual components together at temperatures which correspond to the surrounding, ambient temperature. It is, however, also possible to carry out reactions, such as polymerization, condensation or reaction at reactive groups. This then requires the reaction events to be controlled thermally. Such processes are carried out between 0° C. and 150° C. Preferred temperatures are between 10° C. and 120° C. For the sake of simplicity the compositions are prepared under standard atmospheric pressure. It is likewise possible, however, for preparation to take place at superatmospheric pressure up to 20 bar or reduced pressure down to 20 mbar.

The invention further provides a shaped article, sheetlike structure or elastomer that is coated with a composition of the invention.

The composition of the invention serves further as a protective coating of elastomeric moldings or as a topcoat for sheetlike structures coated on one or both sides with elastomeric materials. These sheetlike structures can be films or textiles, especially wovens, formed-loop knits, drawn-loop knits or nonwovens made from synthetic fibers, natural fibers or mineral fibers. Examples of such are injection moldings or extruded moldings made of elastomeric materials such as natural rubber, nitrile rubber, butyl rubber or silicone rubber. Textile supports coated with elastomeric materials, such as conveyor belts, compensators, protective clothing, electrical insulating hoses, electrical insulating mats, coated textiles which can be used for textile constructions, such as tents, awnings, and tarpaulins, following inventive treatment with the topcoat of the invention, exhibit scratch-resistant, dirt-repellant surfaces having a reduced friction coefficient with themselves and with other materials, and also good bondability with silicone rubber adhesives.

The topcoat is applied to silicone-coated textile membranes in coats of 3-50 g/m². Ideal coat thicknesses are 5-15 g/m². The topcoat must on the one hand have sufficient adhesion to the basecoat. On the other hand it must be bondable; in other words, a silicone adhesive must have sufficient adhesion to the topcoat. The adhesion of the coats is measured in accordance, for example, with DIN 53 530 and ought to be at least 150 N/5 cm.

The compositions of the invention can be applied by spraying, brushing, knife coating, by roller imprinting, by screen printing, by immersion or by similar techniques.

With customary silicone rubber surfaces, the subject invention compositions enter into a firm bond. Curing takes place by evaporation of the solvent and subsequent polycondensation. The cure can be accelerated thermally.

The surfaces treated with the topcoats of the invention are dirt-repellant and scratch-resistant and exhibit reduced friction coefficients with respect to themselves and to other materials, such as glass, metal, plastics, wovens, etc. The surfaces normally treated with the topcoats of the invention are surfaces of silicone rubber moldings, including injection moldings, silicone rubber insulating hoses, medical articles, silicone-rubber-coated wovens, nonwovens, felts, films or papers. Important properties of the base material, such as tensile strength, elongation, elasticity, tear propagation resistance, resistance to heat and cold, and to chemicals or light, are unaffected by the surface treatment.

Advantages of the composition of the invention are that the compositions can also be applied to silicone rubber moldings, injection moldings, insulating hoses, etc. The application is therefore not restricted only to coated wovens. The compositions of the invention are composed not only of pure silicone resins but also of copolymers and silicone fractions. This makes it possible to achieve two or more properties, such as dirt repellency, scratch resistance, and reduced frictional resistance, with only one topcoat. The topcoats do not lead to stiffening of the base material, as is the case with the known methods. This is a substantial advantage particularly in the field of coated textiles.

The silicone topcoat of the invention exhibits improved dirt repellency, a smooth, nonblocking surface, a low coefficient of friction, and very good bondability.

The topcoat can be applied in only one operation. Together with the basecoat application, therefore, only two operations are necessary. In the case of coated wovens, formed-loop knits, drawn-loop knits or felts, commercially customary liquid silicone rubbers can be employed as basecoats. There are known processes which, by addition of adhesion promoters, make it possible in this case to achieve sufficient adhesion without a primer.

Besides the condensation-crosslinking, peroxidically crosslinking binder systems described, the silicone particles can also be incorporated into addition-crosslinking silicone binder systems.

### EXAMPLES

In Examples 1-2 below, relating to the preparation of the silicone particles, unless indicated otherwise, a) all amounts are by weight; b) all pressures are 0.10 MPa (abs.); and c) all temperatures are 20° C.

Light Scattering:

Static and dynamic light scattering were measured with a unit consisting, among other components, of a Stabilite™ 2060-11s Kr laser from Spectra Physics, an Sp-86 goniometer from ALV, and an ALV 3000 digital structurator/correlator. The krypton ion laser operated with a wavelength of 647.1 nm.

Sample Preparation:

The samples (organopolysiloxane particles in toluene; the particular concentration range is indicated in the examples) were filtered three times through Millex™ FGS filters (0.2 μm pore size) from Millipore. The measurement temperature in the case of the light scattering experiments was 20° C. The dynamic light scattering measurements were carried out as a function of angle, from 50′ to 130′ in 20′ steps; the correlation functions were evaluated using the simplex algorithm. In the case of the static light scattering experiment the angular dependency of the scattered light was measured from 30′ to 140′ in 5′ steps.

### Example 1

Metered into an initial charge of 125 g of water, 3 g of benzethonium chloride and 0.3 g of sodium hydroxide solution (10% strength in water) over the course of 45 minutes and with stirring, were 25.0 g of methyltrimethoxysilane. After a further 5 hours of stirring, 1.2 g of trimethylmethoxysilane were added to 25 g of the resulting suspension, with stirring, and stirring was continued for 10 hours more. The suspension was broken by addition of 50 ml of methanol. The precipitated solid was filtered off, washed 3 times with 30 ml of methanol and taken up in 50 ml of toluene. Following the addition of 1.6 g of hexamethyldisilazane and 10 hours of stirring the product was precipitated with 150 ml of methanol, filtered off and dried under a high vacuum. This gave 1.2 g of a white powder whose relative composition was [(CH₃)₃SiO_{1/2}]_{1.38}[CH₃SiO_{3/2}]_{1.0}. By means of static and dynamic light scattering (solvent toluene; measurement concentration range: 0.5-2 g/l) a hydrodynamic particle radius Rₕ of 10.0 nm and a radius of gyration R_{g} of <10 nm were found. This gives a ρ ratio of <1.0. The molecular weight M_{w} of the monodisperse, spherical particles was found to be 2.0×10⁶. The organopolysiloxane particles are readily soluble in toluene, pentane, cyclohexane, dimethylformamide, tetrahydrofuran, dioxane, diethyl ether, methyl methacrylate, styrene and poly(dimethylsiloxane) of viscosity 35 mPa.s.

### Example 2

Metered into an initial charge of 125 g of water, 3 g of benzethonium chloride and 0.3 g of sodium hydroxide solution (10% strength in water) over the course of 1 hour and with stirring, was a mixture of 13.3 g of methyltrimethoxysilane and 11.7 g of dimethyldimethoxysilane. After a further 10 hours of stirring, 1.2 g of trimethylmethoxysilane were added to 25 g of the resulting suspension, with stirring, and stirring was continued for 10 hours more. The suspension was broken by addition of 50 ml of methanol. The precipitated solid was filtered off, washed 3 times with 30 ml of methanol and taken up in 50 ml of toluene. Following the addition of 1.6 g of hexamethyldisilazane and 10 hours of stirring the product was precipitated with 150 ml of methanol, filtered off and dried under a high vacuum. This gave 2.0 g of a white powder which is composed of [(CH₃)₂SiO_{1/2}], [(CH₃)₂SiO_{2/2}], and [CH₃SiO_{3/2}] units. By means of static and dynamic light scattering (solvent toluene; measurement concentration range: 0.05-2 g/l) a hydrodynamic particle radius Rₕ of 11.7 nm and a radius of gyration R_{g} of <10 nm were found. This gives a ρ ratio of <0.85. The molecular weight M_{w} of the monodisperse, spherical particles was found to be 2.0×10⁶. The organopolysiloxane particles are readily soluble in toluene, tetrahydrofuran, chloroform, cyclohexane, pentane, and methyl methacrylate.

### Example 3

A stirring unit fitted with a distillation facility suitable for separating off water discharged azeotropically, is charged with 94 kg of methyl methacrylate, 94 kg of butyl methacrylate and 313 kg of toluene. Water present is removed azeotropically by heating of the mixture at 105° C. When water is no longer discharged from the mixture, it is cooled to 30° C., and 21 kg of Silan GF 31 (commercial product of Wacker-Chemie GmbH) and 2.1 kg of tert-butyl perethylhexanoate are added. The reaction mixture is heated to reflux, with a marked reaction ensuing at about 100° C. The mixture is held at reflux for 8 hours and cooled to 30° C. 15.8 kg of n-butanol and 10.5 kg of Silan M1-Trimethoxy (commercial product of Wacker-Chemie GmbH) are mixed in with stirring. After 30 minutes of stirring, 720 kg of isopropanol and 180 kg of petroleum spirit having a boiling range of 120-140° C. are added. Stirring continues for 30 minutes more. The product is dispensed through a filter, providing a clear, colorless solution having a viscosity of 8 mPa.s and a solids content of 14% by weight.

Subsequently 239 kg of silicone elastomer particles in organic solvents (trade name MIBK 444660 of Wacker-Chemie GmbH) are added. After a further 2 h of stirring at ambient temperature, the product is dispensed into appropriate drums. The clear, colorless liquid exhibits a viscosity of 13 mm²/s and a solids content of 22%.

### Example 4

A unit fitted with a dissolver disk is charged with 47.8 kg of petroleum spirit (boiling range 140-150° C.), 102.6 kg of methyl ethyl ketone, 236.2 kg of xylene, 9.9 kg of n-butanol and 60 kg of tetrahydrofuran and in this solvent mixture 52 kg of Vinnol E 15/40 A (commercial product of Wacker-Chemie GmbH) are dissolved with vigorous mixing.

0.5 kg of isocyanatopropyltriethoxysilane is added and the mixture is boiled at reflux (about 64° C.) for one hour. It is cooled to 30° C. and 50 kg of tetrahydrofuran, 350 kg of toluene and 1000 kg of acetone are added. Intensive mixing takes place for 30 minutes. The clear, colorless product has a viscosity of 11 mPa.s and a solids content of about 2.8% by weight.

Subsequently 239 kg of silicone elastomer particles in organic solvents (trade name MIBK 444660, Wacker-Chemie GmbH) are added. After a further 2 h of stirring at ambient temperature the product is dispensed into appropriate drums. The clear, colorless liquid exhibits a viscosity of 13 mm²/s and a solids content of 22%.

### Example 5

A stirrer mechanism with top-mounted distillation unit is charged with 700 kg of a methylsilicone resin in toluene solution (commercially available as silicone resin solution K toluene (Wacker-Chemie GmbH) and 200 kg of silicone resin solution K0118 (Wacker-Chemie GmbH), and these components are mixed. With continual stirring, 324 kg of toluene are distilled off under atmospheric pressure by heating.

The unit plus contents is cooled to room temperature and 108 kg of methyltriethoxysilane (trade name M1-Triethoxysilane of Wacker-Chemie GmbH), 54 kg of tetraethoxysilane (trade name TES28, Wacker-Chemie GmbH), 54 kg of vinyltriethoxysilane (trade name Geniosil GF56, Wacker-Chemie GmbH) and 5 kg of zirconium butoxide are added in the stated order with stirring.

Stirring is carried out at ambient temperature for 1 h and then 900 kg of acetone and 44 kg of water are added with stirring, and the mixture is stirred for 1 h. Thereafter 239 kg of silicone elastomer particles in organic solvents (trade name MIBK 444660, Wacker-Chemie GmbH) are added. After a further 2 h of stirring at ambient temperature the product is dispensed into appropriate drums. The clear, colorless liquid exhibits a viscosity of 13 mm²/s and a solids content of 22%.

Test Methods for Testing the Dirt Repellency:

There is no generally accepted standard for determining the dirt repellency of silicone-coated membranes. Therefore an internal test method has been developed. Carbon black powder is applied to the membrane at 3 sites (A, B, and C) alongside one another using a paper towel, with 3 circular motions in each case. Circles B and C are then rinsed off with distilled water for 10 s. Circle C is additionally cleaned with a moistened paper towel in 3 circular motions. Circle A shows what quantity of carbon black is taken up by the membrane (dirt pickup). Circle B shows how much carbon black is rinsed off by water (washoff). Circle C shows how much carbon black can be removed by subsequent wet cleaning (cleaning). The dirt pickup is evaluated visually on the basis of a scale from 1 (very low) to 6 (very high). Hence a classification consisting of 3 figures is obtained. The objective of development was to achieve a rating of 2-3-2 or better.

Test Methods for Testing the Bondability:

The bondability is determined by bonding 2 silicone-coated membranes, treated with silicone topcoat, using a silicone adhesive tape (e.g., a tape of Elastosil R 4001/40, trade name of Wacker-Chemie GmbH, thickness 0.6 mm) in a heated press at 180° C. in 2 minutes. The adhesion of the adhesive bond is measured by a peel test in accordance with DIN 53 530. The objective of development was to achieve an overall ply adhesion of 150 N/5 cm or better.

### Example a

The topcoat of the invention is blended with 5% by weight of hydrodimethylpolysiloxane (trade name Vernetzer W [crosslinker], Wacker-Chemie GmbH) and applied by the knife coating method to a glass woven with double-sided silicone rubber coating of Elastosil R 401/40 (trade name of Wacker-Chemie GmbH). The total weight of the coated woven is 240 g/m². Curing the topcoat at 180° C. in 2 minutes gives a topcoat coatweight of 10 g/m².

The membrane without topcoat has a dirt repellency of 5-5-6. On bonding with a silicone adhesive tape, adhesion values of 217 N/5 cm are produced. With state-of-the-art topcoats, dirt repellencies of between 4-4-3 and 2-3-2 are achieved, and adhesion values of between 0 and 106 N/5 cm. The membrane with the topcoat of the invention has a dirt repellency of 2-2-1 and adhesion values of 233 N/5 cm. The dirt pickup results and also the adhesion values are also lowered only slightly by outdoor weathering for 6 months.

The enlarged surface area in conjunction with effective incorporation into the matrix produces good bondability.

| | | | | Adhesion (N/5 cm) |
|---|---|---|---|---|
| | | | | with silicone |
| | A | B | C | adhesive tape |
| silicone-coated membrane | 5 | 5 | 6 | 217 |
| without topcoat | | | | |
| topcoat of EP 718 355 | 3 | 3 | 3 | 106 |
| commercially available silicone | 4 | 4 | 3 | 10 |
| topcoat A | | | | |
| commercially available silicone | 2 | 3 | 2 | 0 |
| topcoat B | | | | |
| inventive topcoat example 5 | 2 | 2 | 1 | 233 |

The results of measurement show that the topcoat of the invention has a good dirt repellency and also good cleaning performance. The adhesion values in the bonding test are markedly higher than the required value.

### Example b

A polyester woven with a base weight of 100 g/m² is provided on both sides with a silicone coating comprising liquid silicone rubber (coatweight 100 g/m). The coated woven without topcoat exhibits dirt pickup scores of 5-4-4. The coated woven with topcoat exhibits dirt pickup scores of 3-3-2. The improvement in dirt pickup behavior is retained to a marked extent even after 5 laundering cycles at 60° C.

### Example c

A nylon woven coated with 30 g/m² Elastosil LR 6250F (trade name of Wacker-Chemie GmbH) is coated with the topcoat of the invention. The topcoat coatweight is 5 g/m².

In order to measure the coefficient of friction, two samples are placed in each case coating to coating. The coefficient of friction is measured in accordance with DIN 53375.

The topcoat of the invention improves the static and kinetic friction coefficient. The scrub test of DIN ISO 5981 shows that the abrasion resistance of the coating is not adversely affected.

| | Friction coefficient | Friction coefficient | |
|---|---|---|---|
| | kinetic | static | Scrub |
| coated nylon woven | 0.35 | 0.42 | >1000 |
| coated nylon | 0.27 | 0.32 | >1000 |
| woven + topcoat | | | |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A composition prepared from polymer components (1) comprising:
(A1) polyorganosiloxanes comprising units (T units) of the formula (RSi—O_{3/2}) and optionally units (M units) of the formula (R₃Si—O_{1/2}) and/or
(A2) polyorganosiloxanes comprising units (Q units) of the formula (Si—O_{4/2}) and optionally units (M units) of the formula (R₃Si—O_{1/2}) in which
R are identical or different optionally halogenated hydrocarbon radicals having 1-18 carbon atoms per radical or are OR¹,
where
R¹ are identical or different, and are hydrogen or a monovalent, optionally substituted hydrocarbon radical having 1-8 carbon atom(s),
with the proviso that per molecule there are 0.01% to 3.0% by weight of Si-bonded radicals OR¹,
and also, optionally, one or more polymer components selected from the group consisting of:
(B) vinyl chloride-hydroxypropyl acrylate copolymers,
(C) vinyl acetate-ethylene copolymers,
(D) polyvinyl chloride,
(E) polyamides,
(F) polyesters,
(G) acrylate-polyester copolymers,
(H) polyamide-polyester copolymers,
(I) vinyl acetate-polyester copolymers, and
(J) monomeric (meth)acrylates, with the proviso that the monomeric (meth)acrylates are copolymerized with silanes containing Si-bonded (meth)acrylate groups,
(2) at least one silane of the general formula
R³ₓSi(OR²)₄₋ₓ
where R² is a monovalent, optionally substituted hydrocarbon radical,
R³ is a monovalent organic radical,
x is 0 or 1,
(3) silicone particles which are crosslinked organopolysiloxane particles which are composed of a single molecule and which have an average diameter of 5 to 200 nm, with at least 80% of the particles possessing a diameter which deviates by not more than 30% from the average diameter, these particles being soluble to an extent of at least 5% by weight in a solvent,
(4) optionally, solvent,
(5) optionally, catalyst, and
(6) optionally, water.

2. The composition of claim 1, wherein in the polyorganosiloxanes (A1) the ratio of M units to T units is 0-1.8:1 and in the polyorganosiloxanes (A2) the ratio of M units to Q units is 0.00-2.7:1.

3. The composition as claimed in claim 2, wherein the crosslinked organopolysiloxane particles composed of a single molecule comprise
0.5% to 80.0% by weight of units of the formula
[R⁴₃SiO_{1/2}] (1),
0 to 99.0% by weight of units of the general formula
[R⁴₂SiO_{2/2}] (2),
0 to 99.5% by weight of units of the formula
[R⁴SiO_{3/2}] (3),
0 to 80.0% by weight of units of the formula
[SiO_{4/2}] (4), and
0 to 20.0% by weight of units of the general formula
[R⁴ₐSi(O_{(3-a)/2})—R⁵—X—(R⁵—Si(O_{(3-a)/2}))_{b}R⁴ₐ] (5),
where
R⁴ are hydrogen atoms or identical or different monovalent SiC-bonded, optionally substituted C₁ to C₁₈ hydrocarbon radicals,
R⁵ are identical or different divalent SiC-bonded, optionally substituted C₁ to C₁₈ hydrocarbon radicals, optionally interrupted by divalent radicals attached on both sides to carbon atoms and selected from the group consisting of —O—, —COO—, —OOC—, —CONR⁶—, —NR⁶CO—, and —CO—,
R⁶ are a hydrogen atom or a radical R⁴,
X is a radical selected from the group consisting of —N═N—, —O—O—, —S—S—, and —C(C₆H₅)₂—C(C₆H₅)₂—,
a denotes the values 0, 1 or 2, and,
b denotes the values 0 or 1,
with the proviso that the sum of the units of the formulae (3) and (4) is at least 0.5% by weight.

4. A process for preparing the composition of claim 1, which comprises reacting or mixing the components of the composition.

5. A shaped article, sheetlike structure or elastomer wherein the shaped article or the elastomer is coated with a composition of claim 1.
